(19) United States
(12) Patent Application Publication
(10) Pub. No.: US 2023/0414456 A1
(43) Pub. Date: Dec. 28, 2023

(54) MEDICATION MONITORING SYSTEMS AND ASSOCIATED METHODS AND DEVICES

(71) Applicant: Quio Technologies LLC, Brooklyn, NY (US)

(72) Inventors: Alexander Dahmani, New York, NY (US); Jared Schwartzentruber, New York, NY (US)

(21) Appl. No.: 18/253,063

(22) PCT Filed: Nov. 22, 2021
(86) PCT No.: PCT/US2021/060320

§ 371 Date: May 16, 2023

Related U.S. Application Data
(60) Provisional application No. 63/117,214, filed on Nov. 23, 2020.

(51) Int. Cl. A61J 7/04; G16H 20/10; G16H 40/67; B65D 1/22; B65D 43/16; B65D 25/06; B65D 43/22; B65D 65/40; B65D 81/00; B65D 25/16

(52) U.S. Cl. CPC A61J 7/0418; G16H 20/10; G16H 40/67; A61J 7/0436; A61J 7/0481; B65D 1/22; B65D 43/16; B65D 25/06; B65D 43/22; B65D 65/40; B65D 81/00; B65D 25/16; A61J 2200/70; B65D 2251/1016; B65D 2313/04; B65D 2543/00194; B65D 2543/00268; B65D 2543/00314

(57) ABSTRACT

Devices, systems, and methods for a medication monitoring system are described. An example medication monitoring system for a user includes a container subassembly comprising one or more compartments, each compartment being configured to store medical equipment or medications prescribed to the user, and a lid of the container subassembly pivotally mounted on a first edge of the container subassembly and comprising a magnet at a second edge opposite to the first edge, and an electronics subassembly comprising a magnetic sensor coupled to the magnet in the lid of the container subassembly, and a communication module configured to communicate with a patient record comprising one or more medication regimens for the user.

### CROSS-REFERENCE TO RELATION APPLICATION

This patent application claims priority to U.S. Provisional Patent Application 63/117,214 which was filed on Nov. 23, 2020, the entire contents of which are incorporated herein by reference and relied upon.

### TECHNICAL FIELD

The present technology relates to medication monitoring systems and associated methods and devices.

### BACKGROUND

There are many marketed medication reminder devices available today. Such conventional devices are built to attach or replace specific medication delivery protocols or techniques. This is under the assumption that, the more data collected and the closer that data is to the time of medication administration, the more actionable interventions can be achieved.

This approach, however, often compromises the user experience by adding extra steps to medication regimens, requiring devices to manage Bluetooth® connectivity, charging the device, and adding/removing the device (for delivery device add-ons). In addition, these devices are often medication-specific, so each new underlying medication or medication delivery device requires a completely new set of hardware and sensors to detect the desired actions. Existing implementations for medication monitoring are typically produced in smaller volumes (to address many niche markets), have high unit prices and, depending on the application, must comply with additional regulatory requirements (depending on the effect of the medication or delivery device).

### SUMMARY

Embodiments of the disclosed technology relate to medication monitoring systems and their use for medication regimen adherence. An example medication monitoring system is a low-cost, general use system that is configured to support and work with a wide array of medications. It employs a flexible platform with a fixed set of sensors and wireless connectivity to interface with several types of medicaments, and leverages a platform design for universal applicability, a battery that will last the lifetime of the device, and a communication module that comes pre-paired to the patient's account to ensure seamless delivery of data without the need to manage or charge the device.

The disclosed embodiments include systems, devices, and methods for medication monitoring and medication regimen adherence. An example medication monitoring system for a user includes a container subassembly comprising one or more compartments, each compartment being configured to store medical equipment or medications prescribed to the user, and a lid of the container subassembly pivotally mounted on a first edge of the container subassembly and comprising a magnet at a second edge opposite to the first edge, and an electronics subassembly comprising a magnetic sensor coupled to the magnet in the lid of the container subassembly, and a communication module configured to communicate with a patient record comprising one or more medication regimens for the user.

### BRIEF DESCRIPTION OF THE DRAWINGS

Many aspects of the present technology can be better understood with reference to the following drawings. The components in the drawings are not necessarily drawn to scale. Instead, emphasis is placed on illustrating the principles of the present technology. Components may also be shown schematically.

FIGS. 1A and 1B show an example of a medication monitoring system configured in accordance with the present technology in an open position and a closed position, respectively.

FIGS. 2A and 2B show another example of a medication monitoring system configured in accordance with the present technology in an open position and a closed position, respectively.

FIGS. 3-5 show additional examples of medication monitoring systems configured in accordance with the present technology.

FIGS. 6A and 6B show yet another example of a medication monitoring system that can accommodate a pill organizer and is configured in accordance with an embodiment of the present technology.

FIG. 7A shows an example of an electronics subassembly of a medication monitoring system in accordance with an embodiment of the present technology.

FIGS. 7B and 7C show examples of a printed circuit board (PCB) configured in accordance with embodiments of the present technology.

FIG. 8 is a flowchart of a method for using a medication monitoring system in accordance with embodiments of the present technology.

FIG. 9 is a flowchart of a method for deploying a medication monitoring system in accordance with embodiments of the present technology.

FIG. 10 shows an example of a hardware platform that can implement some methods and techniques associated with the present technology.

### DETAILED DESCRIPTION

### A. Overview

The present technology is directed to medication monitoring systems and associated methods and devices. The disclosed medication monitoring systems are low-cost, general use systems that can be configured to support and work with a wide array of medications. The disclosed systems employ a flexible platform with a fixed set of sensors and wireless connectivity to interface with, for example, injectables (e.g., pen injectors, syringes, and vials), inhaler medications (e.g., relievers, preventers, long-acting bronchodilators), and pill medications (e.g., pill bottles, pill organizers), or any combination thereof. The disclosed medication monitoring systems are expected to overcome the above-referenced drawbacks with conventional medication reminder devices by leveraging a platform design for universal applicability, a battery that will last the lifetime of the device, and a communication module that comes pre-paired to the patient's account to ensure seamless delivery of data without the need to separately manage or repeatedly charge the device.

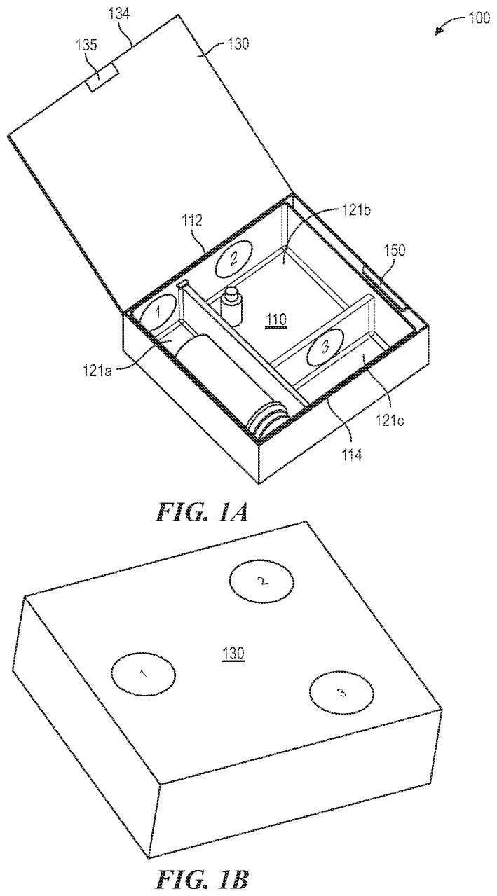

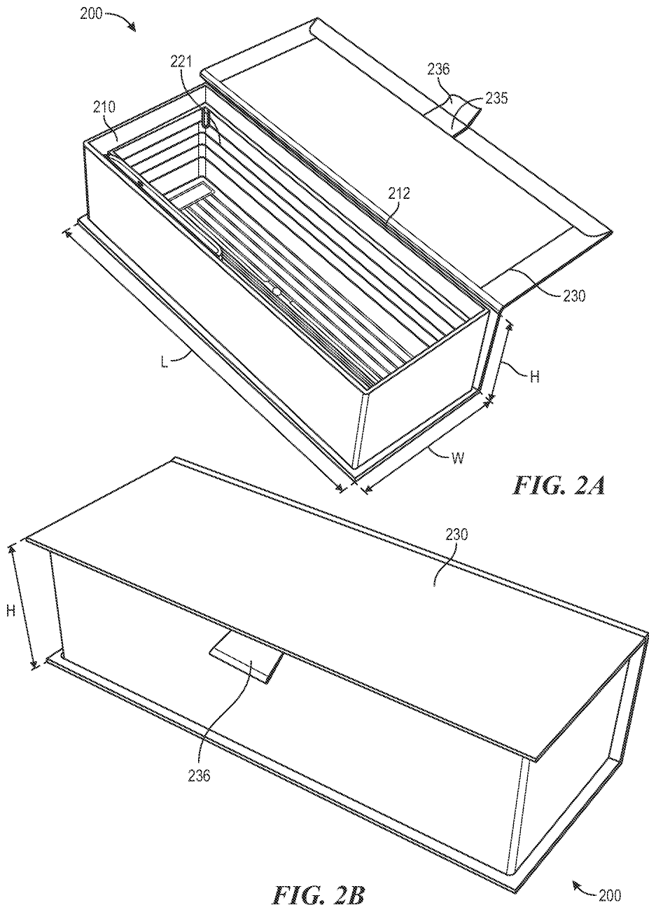

Specific details of various embodiments of the present technology are described below with reference to FIGS. 1-10. Although many of the embodiments are described below with respect to medication monitoring systems and associated methods, other embodiments are within the scope of the present technology. Additionally, other embodiments of the present technology can have different configurations, components, and/or procedures than those described herein.

### B. Embodiments of Medication Monitoring Systems

FIGS. 1A and 1B show an example of a medication monitoring system 100 configured in accordance with the present technology in an open position and a closed position, respectively. As shown in FIG. 1A, the medication monitoring system 100 comprises a container subassembly 110 including one or more compartments 121. In the illustrated embodiment, for example, the container subassembly 110 comprises a first compartment 121a, a second compartment 121b, and a third compartment 121c. In other embodiments, the container subassembly 110 may include a different number of compartments 121. For example, the container subassembly 110 may include a single compartment or more than three compartments. In additional embodiments, the container subassembly 110 may include two compartments, which may or may not be of equal sizes. Each compartment 121a-c in the container subassembly 110 can be configured to store medical equipment or medications prescribed to the user.

The system 100 further comprises a lid 130 pivotably mounted to a first edge 112 of the container subassembly 110. The lid 130 is pivotably movable between an open position (FIG. 1A) in which the compartment(s) 121 are accessible and a closed position (FIG. 1B) in which the compartment(s) 121 are inaccessible. In the illustrated embodiment, the lid 130 includes a magnet 135 at or near a center of an edge 134 opposite to the edge of the lid 130 that is pivotally mounted to the container subassembly 110.

In some embodiments, the container subassembly 110 can be made from paper, cardboard, or paperboard. In other embodiments, paper, cardboard, or paperboard can be laminated in a latex material for durability and water resistance. In still further embodiments, the container subassembly 110 can be made of other suitable materials.

In some embodiments, the one or more compartments 121 of the container subassembly 110 can include a liner. Such a liner can enable a specific medicament to be securely stored in the corresponding compartment 121. The liner is an optional component that may not be included in some embodiments. In some embodiments, the one or more compartments 121 can further include a plastic insert to provide a disinfectable surface for medications where bodily fluids may come into contact, and further include partition(s) so the user can organize their belongings. In these embodiments, the inserts and/or internal liners can be formed using the laminated paper, cardboard, or paperboard, or using vacuum-formed glycol-modified polyethylene terephthalate (also known as PETG®). In other embodiments, other plastic materials and other plastic molding methods (e.g., injection molding) may be used to form the inserts and/or internal liners. In yet other embodiments, nylons, co-polyesters (e.g., Fillamentum® CPE), or polycarbonates can be used in conjunction with 3D printing methods to form the inserts and/or internal liners.

In some embodiments, the medication monitoring system 100 can further include an electronics subassembly 150, which can be placed in between the container subassembly 110 and the outer wall of the medication monitoring system 100. The electronics subassembly 150 can include a magnetic sensor that is coupled to the magnet 135 in the lid 130 of the container subassembly, and a communication module configured to communicate remotely with a patient record comprising a medication regimen for the user. As noted previously, for example, FIG. 1B shows the medication monitoring system 100 in a closed position, and in which the magnet 135 carried by lid 130 is now physically coupled to the magnetic sensor in the electronics subassembly 150. The electronics subassembly 150 will be described in further detail below in Section C.

FIGS. 2A and 2B show another example of a medication monitoring system 200 configured in accordance with the present technology. FIG. 2A illustrates the system 200 in an open position, and FIG. 2B illustrates the system 200 in a closed position. The medication monitoring system 200 shown in FIGS. 2A and 2B has a container subassembly 210 with a single compartment 221, which can be used to store an inhaler or a single medical device (e.g., an insulin pen injector) or a pill organizer. The system 200 further comprises a lid 230 pivotably mounted to a first edge 212 of the container subassembly 210. In this embodiment, a magnet 235 carried by the lid 230 can be attached to a fabric tab 236 that enables the user to easily open the lid 235 of the container subassembly 210 to access the medicament therein. The system 200 of FIG. 2A further includes an insert 221 that may be molded to fit the specific medicament being stored in the system 200.

As shown in FIG. 2A, the medication monitoring system 200 can have a first exterior dimension (e.g., length, L), a second exterior dimension (e.g., width, W), and a third exterior dimension (e.g., height, H). The interior dimensions of the medication monitoring system 200 are substantially similar to the exterior dimensions (accounting for the thickness of the container subassembly 210) and can be customized for specific medicaments.

In an example, the system 200 can be configured to store an insulin kit, such that the interior dimensions of the container subassembly can range from 9.0″-9.5″ (length in inches)×3.0″-3.5″ (width in inches)×2.0″-2.5″ (height in inches).

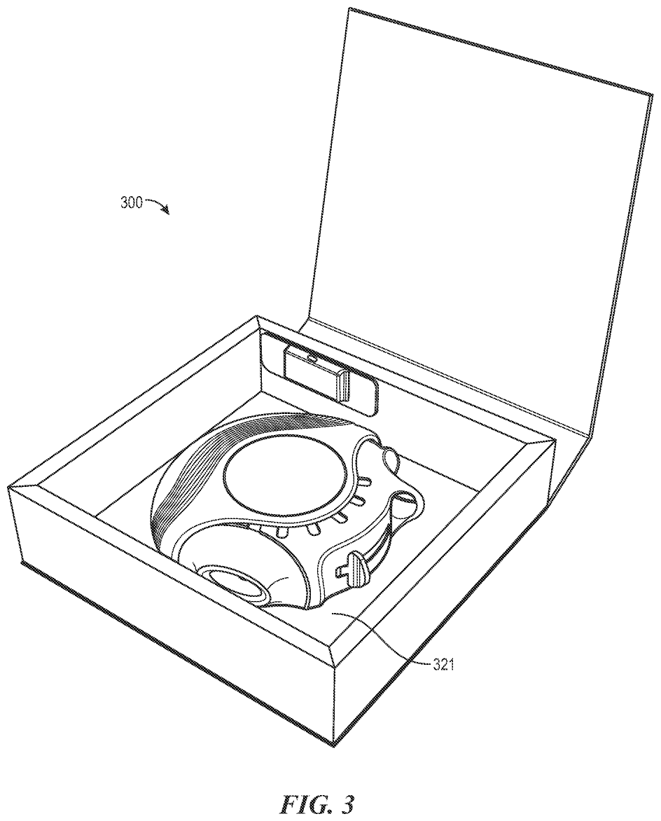

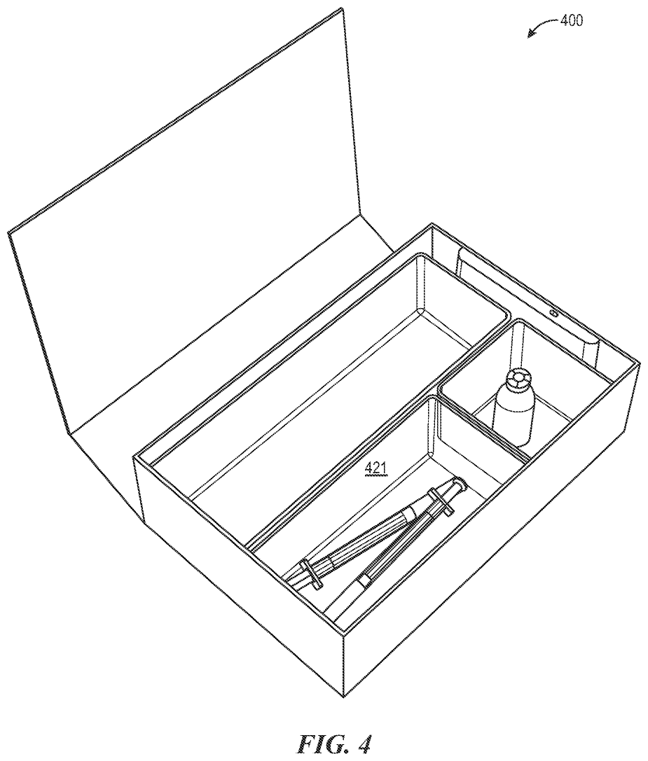

In another example, as shown in FIG. 3, the system 200 can be configured to store an inhaler, such that interior dimensions of the subassembly can range from 3.0″-4.0″ (length in inches)×3.5″-4.0″ (width in inches)×2.0″-2.5″ (height in inches).

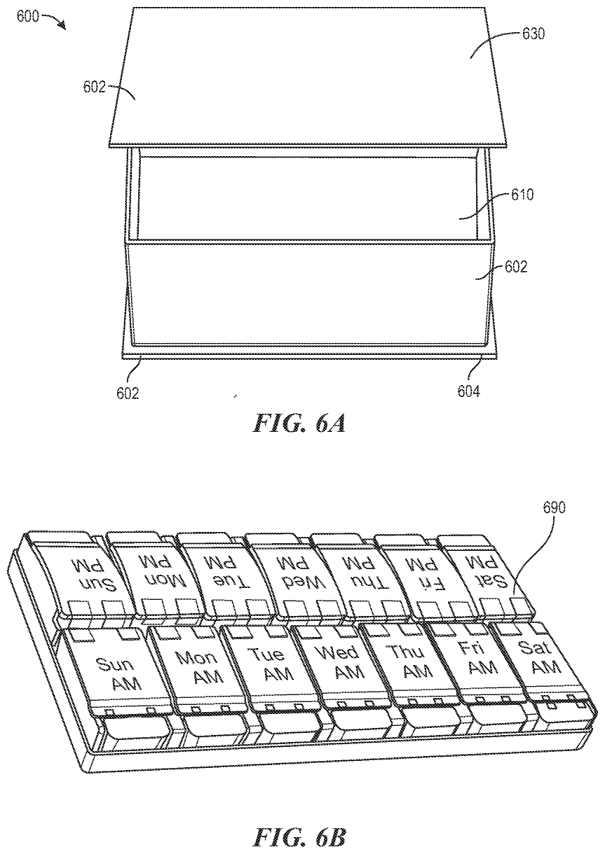

In yet another example, as shown in FIGS. 6A and 6B, the system 200 can be configured to store a pill organizer, such that interior dimensions of the subassembly are 5.0″-10.0″ (length in inches)×2.0″-5.0″ (width in inches)×1.0″-4.0″ (height in inches). It will be appreciated that the above examples are merely specific implementations associated with particular medicaments, and that the systems described herein can have a variety of different sizes/shapes depending upon the selected medicaments.

In the embodiment shown in FIGS. 2A and 2B, the container subassembly 210 is composed of a rigid magnetic cardboard box. The rigid magnetic cardboard box can be built quickly, with various sizes and colors, at a low cost, and in small volumes. This deployment paradigm, which is further detailed in Section D, allows for a multitude of stock keeping units (SKUs) that leverage the same electronics subassembly. In other embodiments, however, the container subassembly 210 may be composed of other suitable materials. Thus, the described embodiments solve the problem of existing systems being medication-specific, and being produced in smaller volumes with higher unit costs.

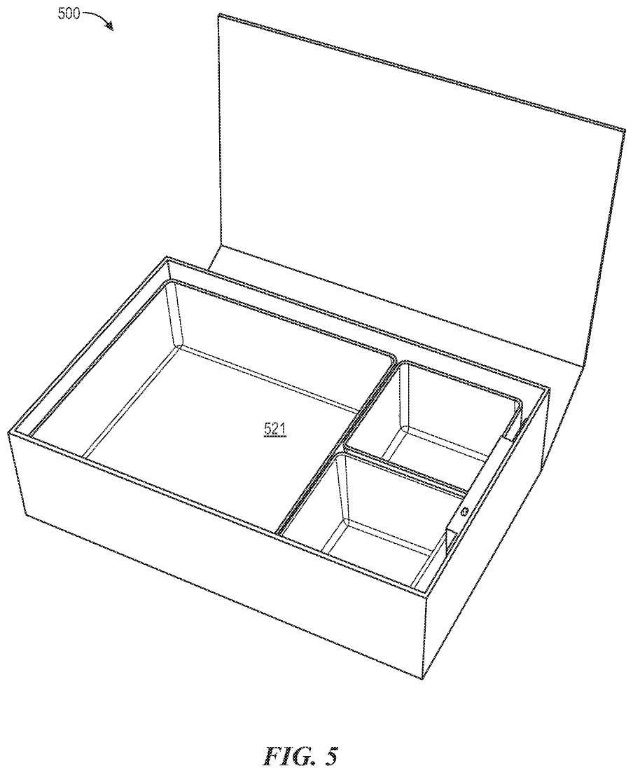

FIGS. 3-5 show additional examples of medication monitoring systems configured in accordance with the present technology. FIG. 3, for example, shows yet another example of a medication monitoring system 300 with a single compartment 321, which is configured to store a Diskus inhaler. FIG. 4 shows yet another example of a medication monitoring system 400, which a patient can use to store syringes, vials, alcohol swabs and a blood lancet, all organized in the three compartments 421 of the same medication monitoring system 400. FIG. 5 shows yet another example of a medication monitoring system 500 with three compartments 521 that are configured differently from the example shown in FIG. 4.

FIGS. 6A and 6B show yet another example of a medication monitoring system 600 configured in accordance with an embodiment of the present technology. The system 600 is sized and shaped to accommodate a pill organizer 690 (FIG. 6B). As shown in FIG. 6A, the system 600 has a main exterior portion 602 that includes a lid 630 and a base portion 604, and a secondary exterior portion 606 that includes side walls 608 of the medication monitoring system 600. The main and secondary exterior portions 602 and 604, which are made of a low-cost material that can be customized for each user, define the container subassembly (or interior) 610 that includes a compartment to hold the pill organizer 690 and/or other selected medicaments associated with a medication regimen of the user. In other embodiments, the system 600 may include one or more additional compartments to hold additional medicaments or other items of the user.

In this example, an electronics subassembly (not shown) of the system 600 can be configured to monitor a 14-day pill regimen of the user. That is, the system 600 can ensure that the user adheres to the medication regimen that has been prescribed using the electronics subassembly. Further details regarding the electronics subassembly are discussed in the following section.

### C. Electronic Subassembly of the Medication Monitoring System

The electronics subassembly of a medication monitoring system is a self-contained unit that includes a communication module, a usage sensor, and a battery, and is configured to interface with any medication monitoring system to enable a user to adhere to a medication regimen. In some embodiments, the electronics subassembly further includes an encasement and/or a double-sided adhesive backing. The adhesive backing is used to adhere the electronics subassembly to an accompanying box, and the electronics sensors are aligned with an adjacent magnet in the box.

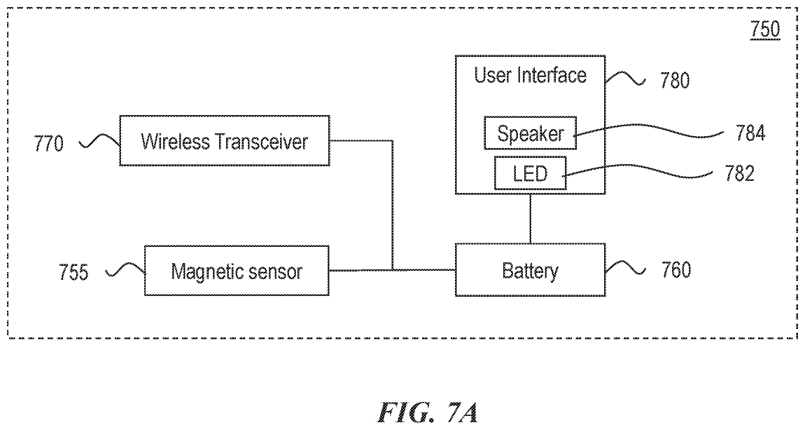

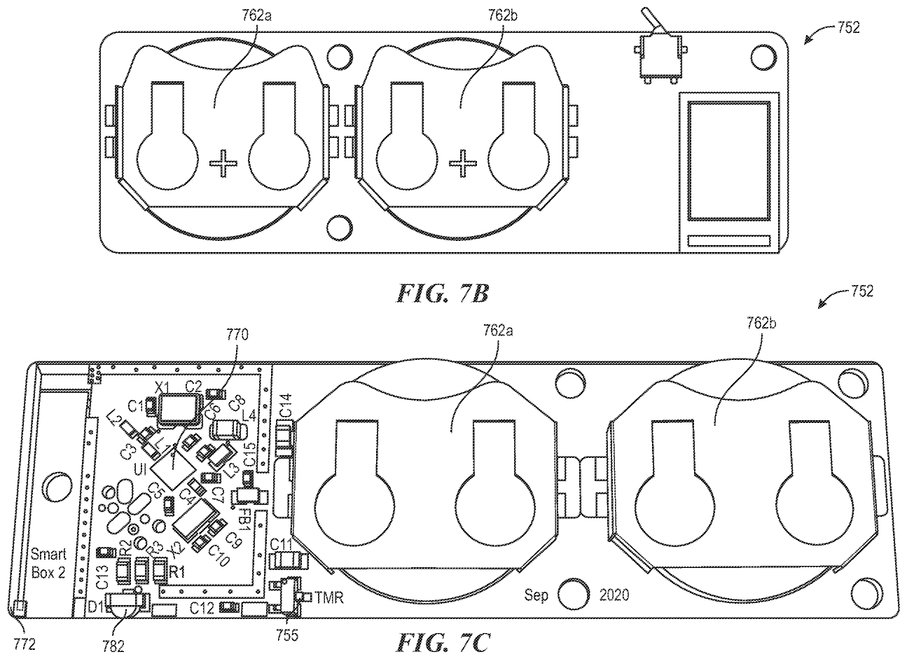

FIG. 7A shows an example of an electronics subassembly 750 configured in accordance with an embodiment of the present technology. As shown therein, the electronics subassembly 750 includes a battery 760 coupled to and providing power to a magnetic sensor 755 (e.g., the usage sensor), a wireless transceiver 770 (e.g., the communication module), and a user interface 780.

In some embodiments, the communication module 770 may either be Bluetooth®, Wi-Fi®, Zigbee®, cellular, or another (preferably low power) communication protocol. The battery 760 may be selected based on the desired communication protocol, since different communication protocols require different batteries to support operation. In an example, the battery 760 is sized to accommodate 1-2 years of operation without the need to recharge.

In some embodiments, the magnetic sensor 755 can be a reed switch, a TMR sensor, a Hall-effect sensor, or the like. The magnetic sensor 755 couples with a magnet assembled within the lid of the rigid cardboard box (e.g., magnet 135 in lid 130 in FIG. 1A). In an example, the magnet 135 within the lid 130 is a permanent magnet. In another example, the magnets and metal shims can be placed in cutouts and encased in the laminate of the paper, cardboard, or paperboard.

In some embodiments, the user interface 780 can be configured to inform the user of the status of the electronics subassembly 750, including one or more states (e.g., event detected, error, low battery, and the like). For an example, the user interface 780 can include a light-emitting diode (LED) 782 and/or a speaker 784, which can be used to provide the user with status information, or one or more reminders based on a medication regimen of the user and a current time.

In some embodiments, the electronic subassembly 750 is configured to generate an event and log the event in the patient record upon a determination that the lid of the container subassembly was opened.

In some embodiments, the electronics subassembly 750 comprises a unique identifier to associate the electronics subassembly with the container subassembly. In an example, the unique identifier is a Medium Access Control (MAC) ID, or an International Mobile Equipment Identity (IMEI) associated with a cellular service.

In some embodiments, the electronics subassembly 750 can be built around a printed circuit board (PCB) 752, examples of which are shown in FIGS. 7A and 7B. As shown therein, for example, the PCB includes button cell receptacles (762A and 762B), the magnetic sensor 755, a user interface (e.g., LED 782), a microcontroller or a microprocessor, and a communications module 770 coupled to an antenna 772.

### D. Deployment of the Medication Monitoring System

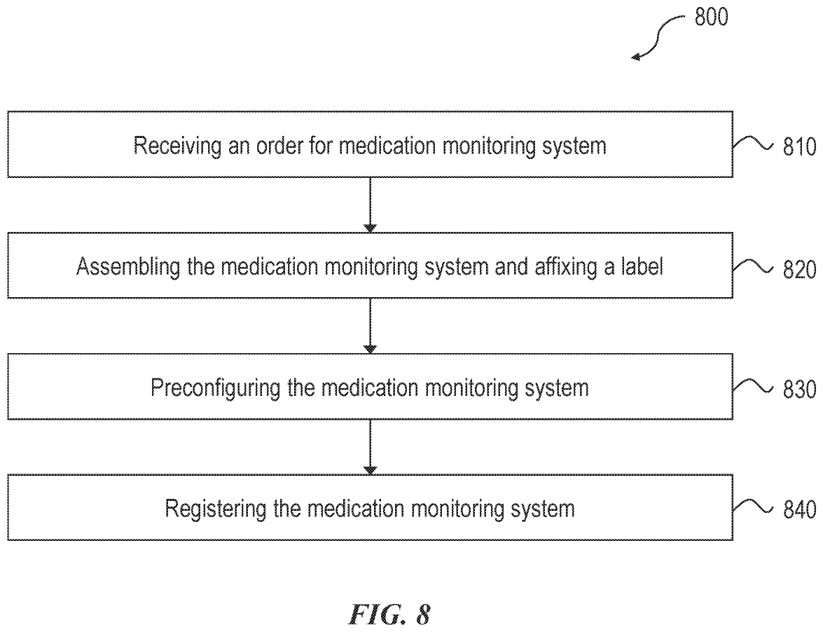

Some embodiments of the disclosed technology are directed to the deployment of the medication monitoring system. FIG. 8, for example, is a flowchart of a method 800 for using a medication monitoring system in accordance with embodiments of the present technology. The method 800 may be utilized with any of the systems disclosed herein or other suitable medication monitoring systems.

The method 800 includes, at operation 810, receiving an order for a medication monitoring system. In some embodiments, the order for the medication monitoring system includes a first sub-order for the container (e.g., the rigid cardboard box) and a second sub-order for the electronics subassembly. Upon receiving the order, a nominal (or generic) medication monitoring system is associated with an end user's regimen or regimens.

The method 800 includes, at operation 820, assembling the medication monitoring system and affixing a label. In some embodiments, to ensure flexibility with the design, the final assembly (of the electronics subassembly and rigid cardboard box) is completed at the point of distribution. In an example, the various boxes and electronics subassemblies can be warehoused separately, and when the order is placed, the units are combined and shipped to the end-user. In other embodiments, the final assembly can be completed (for standard types or configurations of medicaments) prior to receiving the order for the medication monitoring system.

In some embodiments, and based on the regimen(s) (medications prescribed, auxiliary supplies needed, etc.), the nominal medication monitoring system is configured with the appropriate dimensions. The box and electronics subassembly SKUs are transmitted to the warehouse for fulfillment, where the two SKUs are then picked and assembled.

Continuing with operation 820, the box SKU identifier is scanned and associated with the order to confirm the correct box was selected. The electronics subassembly unique identifier (e.g., barcode, QR code, or a similar machine-readable code) is also scanned, associated with the order, and is then paired to the end-user's regimen. This unique identifier can be used to associate the medication monitoring system to a specific end user on the backend cloud platform and/or mobile application.

As part of the final assembly (operation 820), a label may be applied to the outside of the medication monitoring system, which includes information that matches one or more digital regimens in the patient's record. The label is designed to inform the patient which medications should be stored in the medication monitoring system when they receive it. The label also reminds the patient which medications are inside the medication monitoring system prior to opening the lid and generating an event, which is advantageous when a patient has multiple medication monitoring systems for different medications.

The method 800 includes, at operation 830, preconfiguring the medication monitoring system. In some embodiments, if the electronics subassembly supports Bluetooth®, its associated MAC ID (or IMEI) is then pushed from a cloud system to the end user's associated smartphone app or cellular hub, which preconfigures the medication monitoring system. In other embodiments, if the preconfiguration (or fulfillment) operation cannot scan the machine readable code, the hardware ID associated with the medication monitoring system is manually inputted and transmitted to the cloud system, which preconfigures the medication monitoring system.

In some embodiments, and as discussed earlier, no personal or identifying information is stored on the medication monitoring system to ensure the integrity of the data. The user's smartphone or the cloud system manages all the medicament and medication regimen information, patient name, and/or personal identifiable information (PII). This enables the data to be securely disseminated to other third-party providers (e.g., insurance companies or medical service providers) as needed, and only when authorized by the user or their agent. Furthermore, the remote management of medicament and patient information advantageously enables the medication monitoring system to send data without requiring secure pairing, secure transmission of data, or classification as a medical device.

The method 800 includes, at operation 840, registering the medication monitoring system. When the end-user receives the medication monitoring system and it broadcasts for the first time, the device will automatically register and begin sending data to the cloud system without the end user's need to set up and manage the connection.

### E. Regimen Adherence Using the Medication Monitoring System

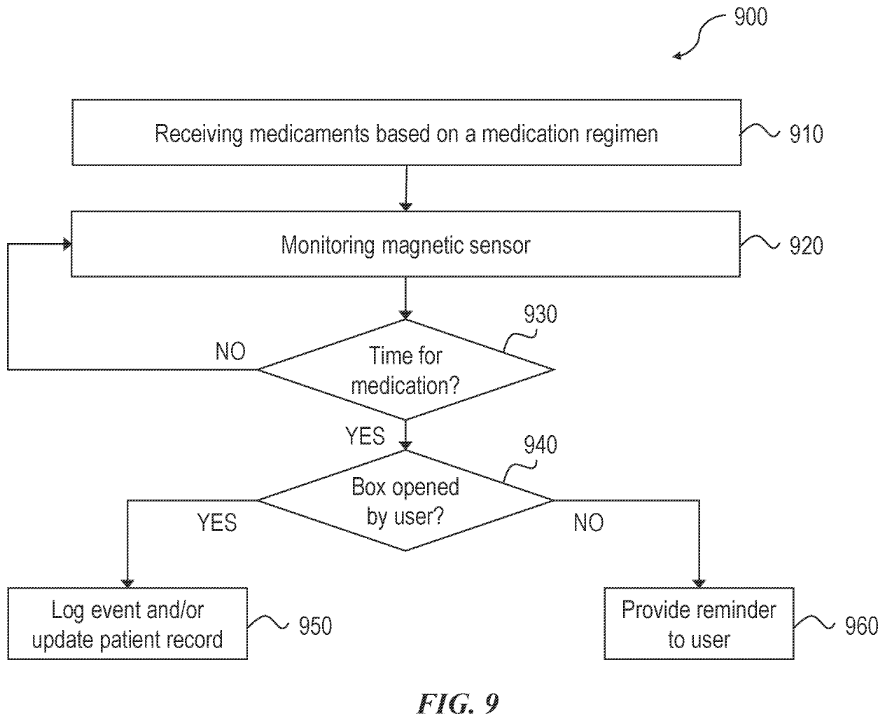

Some embodiments of the disclosed technology are directed to enabling a user to adhere to a medication regimen using a medication monitoring system. FIG. 9 is a flowchart of a method 900 for deploying a medication monitoring system in accordance with embodiments of the present technology. The method 900 may be utilized with any of the systems disclosed herein or other suitable medication monitoring systems.

The method 900 includes, at operation 910, receiving medicaments based on a medication regimen. After the medication monitoring system has been automatically registered (as described above), the end user can then place their medication(s) within the box's cavity and proceed to use the medication monitoring system as a storage container for their medication(s).

The method 900 includes, at operation 920, monitoring the magnetic sensor to determine whether or not the box has been opened.

The method 900 includes, at operation 930, determining whether the current time is a time for medication based on the user's medication regimen. If it is not time for a dose of the medicament stored in the medication monitoring system (the “NO” path from 930), the method goes back to operation 920, wherein the magnetic sensor is monitored. However, if the current time coincides with a time for a dose of the medicament (the “YES” path from 930), then the method proceeds to operation 940. In some embodiments, the determining is performed on the smartphone of the user (or equivalently, the cloud system or hub).

The method 900 includes, at operation 940, determining whether the user has opened the box. When the end user goes to take their medication (the “YES” path from 940), they will lift the lid to the medication monitoring system. This action separates the sensor from the adjacent magnet and triggers the connectivity module to wake up and transmit the event (operation 950). The medication monitoring system may also provide visual indication to the user through an LED that flashes when the box is opened.

However, if the box is not opened by the user (the “NO” path from 940), then a reminder is provided to the user (operation 960). In an example, the speaker or LED on the user interface (e.g., speaker 784 or LED 782 on user interface 780 in FIG. 7) may be used to provide an aural or visual indication to the user, respectively. In another example, the wireless transceiver can be used to send the reminder to the user's smartphone.

In some embodiments, the electronics subassembly (e.g., electronics subassembly 750 in FIG. 7A) is associated with a software system for monitoring medication adherence. The software system stores patient records containing digital medication regimens. Each electronics subassembly is linked to a specific patient record when in use, which results in the data generated by the electronics subassembly to be transmitted to and stored in the associated patient record. Each electronics subassembly is further linked to one or more digital medication regimens in said patient record in the software system. When the electronics subassembly generates an event from the user opening the box, a dose is logged in the patient record in the software system for the one or more digital regimens linked to the electronics subassembly.

Depending on the associated regimen or regimens, the system associates the transmitted event with a medication regimen. For example, if the medication monitoring system is associated with a single regimen, then it automatically associates it with said regimen. But if the medication monitoring system houses a pill organizer with multiple pills at multiple scheduled times per day, the system will assign a time estimate to the event and determine the most appropriate regimen to associate with the captured event.

In some embodiments, the patient can then view their medication history through a mobile application, a patient portal, or through SMS notification messages, depending on their preferences. A remote medical team can also view the patient's medication history in the system or in the patient's chart on an electronic medical record (EMR). In addition, the system can be configured to automatically trigger alerts and notifications for missed or unexpected medication consumption. These alerts are provided to the end user's medical team, and may also be shared with the patient, the patient's accountability partner, and/or the patient's power of attorney/caregiver.

### F. Example Implementations of the Medication Monitoring System

Embodiments of the disclosed technology include a medication monitoring system for a user that includes a container subassembly comprising one or more compartments, each compartment being configured to store medical equipment or medications prescribed to the user, and a lid of the container subassembly pivotally mounted on a first edge of the container subassembly and comprising a magnet at a second edge opposite to the first edge, and an electronics subassembly comprising a magnetic sensor coupled to the magnet in the lid of the container subassembly, and a communication module configured to communicate with a patient record comprising one or more medication regimens for the user.

In some embodiments, the container subassembly is a rigid magnetic cardboard box (e.g., as described in FIGS. 2A, 2B and 3-5, and Section B). In some examples, the rigid magnetic cardboard box is made from paper, cardboard, or paperboard. In some examples, the paper, cardboard, or paperboard is laminated in a latex material.

In some embodiments, the one or more compartments in the container subassembly are configurable based on the one or more medication regimens of the user.

In some embodiments, a size or a shape of at least one of the one or more compartments is configurable based on the one or more medication regimens of the user.

In some embodiments, the electronics subassembly (e.g., as described in Section C) further comprises a user interface configured to provide a status of the electronics subassembly to the user, and a battery configured to provide power to the communication module and the user interface.

In some embodiments, the electronics subassembly is assembled inside a wall of a container subassembly during a container manufacturing process.

In some embodiments, the electronics subassembly is mounted into an electronics case, and wherein the electronics case is rigidly affixed to a wall of the container subassembly.

In some embodiments, the electronic subassembly is configured to generate an event and log the event in the patient record upon a determination that the lid of the container subassembly was opened.

In some embodiments, the electronic subassembly is configured to provide a reminder to the user based on the one or more medication regimen and a current time.

In some embodiments, the electronics subassembly comprises a unique identifier to associate the electronics subassembly with the container subassembly. In some examples, the unique identifier is a Medium Access Control (MAC) ID or an International Mobile Equipment Identity (IMEI).

In some embodiments, and as described in Section B, at least one of the plurality of compartments further comprises a plastic insert to provide a disinfectable surface.

In some embodiments, a liner for each of the one or more compartments or the plastic insert is made from vacuum-formed glycol-modified polyethylene terephthalate (or PETG®).

In some embodiments, the medication comprises an injectable, an inhaler, or a pill, and wherein the medical equipment comprises an alcohol swab, a disinfectant, a bandage, or a lancet.

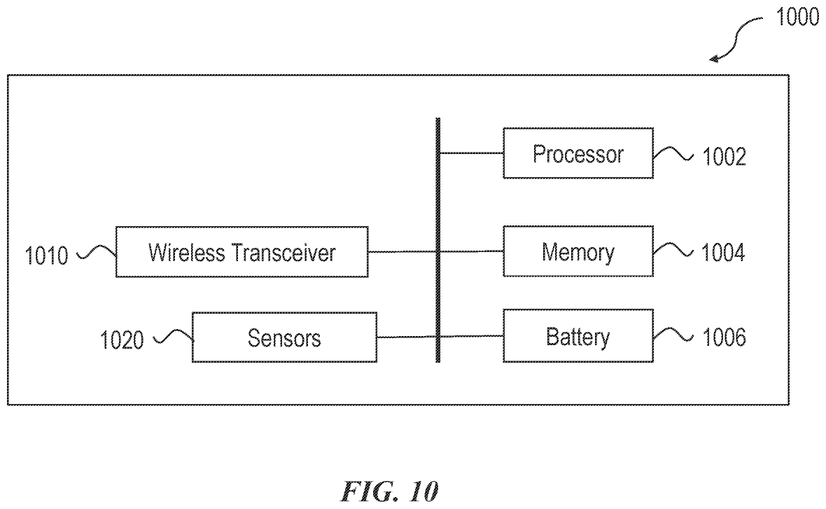

FIG. 10 shows an example of a hardware platform 1000 that can be used to implement some of the techniques associated with the present technology. For example, the hardware platform 1000 may implement method 800 or may implement the various modules described herein. The hardware platform 1000 may include a processor 1002 that can execute code to implement a method. The hardware platform 1000 may include a memory 1004 that may be used to store processor-executable code and/or store data. The hardware platform 1000 may further include a battery 1006. The hardware platform may further include a wireless transceiver 1010 and sensors 1020. In some embodiments, the wireless transceiver 1010 supports Bluetooth®, Wi-Fi®, Zigbee®, cellular, and other similar wireless communication protocols. In some embodiments, some portion or all of the wireless transceiver 1010 may be implemented in the processor 1002. In some embodiments, some portion of the hardware platform 1000 may be implemented as part of the PCB shown in FIGS. 7A and 7B.

### CONCLUSION

Implementations of the subject matter and the functional operations described in this patent document can be implemented in various systems, digital electronic circuitry, or in computer software, firmware, or hardware, including the structures disclosed in this specification and their structural equivalents, or in combinations of one or more of them. Implementations of the subject matter described in this specification can be implemented as one or more computer program products, e.g., one or more modules of computer program instructions encoded on a tangible and non-transitory computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The term “data processing unit” or “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random-access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of nonvolatile memory, media and memory devices, including by way of example semiconductor memory devices, e.g., EPROM, EEPROM, and flash memory devices. The processor and the memory can be supplemented by, or incorporated in, special purpose logic circuitry.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described, and other implementations, enhancements and variations can be made based on what is described and illustrated in this patent document.

1. A medication monitoring system for a user, comprising:
a container subassembly comprising:
one or more compartments, each compartment being configured to store medical equipment or medications prescribed to the user, and
a lid of the container subassembly pivotally mounted on a first edge of the container subassembly and comprising a magnet at a second edge opposite to the first edge; and
an electronics subassembly comprising:
a magnetic sensor coupled to the magnet in the lid of the container subassembly, and
a communication module configured to communicate with a patient record comprising one or more medication regimens for the user.

2. The medication monitoring system of claim 1, wherein the container subassembly is a rigid magnetic cardboard box.

3. The medication monitoring system of claim 2, wherein the rigid magnetic cardboard box is made from paper, cardboard, or paperboard.

4. The medication monitoring system of claim 3, wherein the paper, cardboard, or paperboard is laminated in a latex material.

5. The medication monitoring system of claim 1, wherein the one or more compartments in the container subassembly are configurable based on the one or more medication regimens of the user.

6. The medication monitoring system of claim 1, wherein a size or a shape of at least one of the one or more compartments is configurable based on the one or more medication regimens of the user.

7. The medication monitoring system of claim 1, wherein the electronics subassembly further comprises:
a user interface configured to provide a status of the electronics subassembly to the user; and
a battery configured to provide power to the communicate module and the user interface.

8. The medication monitoring system of claim 1, wherein the electronics subassembly is assembled inside a wall of a container subassembly during a container manufacturing process.

9. The medication monitoring system of claim 1, wherein the electronics subassembly is mounted into an electronics case, and wherein the electronics case is rigidly affixed to a wall of the container subassembly.

10. The medication monitoring system of claim 1, wherein the electronic subassembly is configured to generate an event and log the event in the patient record upon a determination that the lid of the container subassembly was opened.

11. The medication monitoring system of claim 1, wherein the electronic subassembly is configured to provide a reminder to the user based on the one or more medication regimen and a current time.

12. The medication monitoring system of claim 1, wherein at least one of the plurality of compartments further comprises a plastic insert to provide a disinfectable surface.

13. The medication monitoring system of any claim 12, wherein a liner for each of the one or more compartments or the plastic insert is made from vacuum-formed glycol-modified polyethylene terephthalate.

14. The medication monitoring system of claim 1, wherein the medication comprises an injectable, an inhaler, or a pill, and wherein the medical equipment comprises an alcohol swab, a disinfectant, a bandage, or a lancet.

15. The medication monitoring system of claim 1, wherein the electronics subassembly comprises a unique identifier to associate the electronics subassembly with the container subassembly.

16. The medication monitoring system of claim 15, wherein the unique identifier is a Medium Access Control (MAC) ID or an International Mobile Equipment Identity (IMEI).

17. A method for adherence to the one or more medication regimens using the medication monitoring system of claim 1.
